SEQUENCE LISTING (21 sequences)

SEQ ID NO: 1 - DNA, 18 bp, Artificial Sequence
  Probe
tggatcccccgggctgca

SEQ ID NO: 2 - DNA, 30 bp, Artificial Sequence
  Primer
gaaactgtaaaattgactaaataccttgct

SEQ ID NO: 3 - DNA, 26 bp, Artificial Sequence
  Primer
agggttattgaatatgatcggaattc

SEQ ID NO: 4 - DNA, 29 bp, Artificial Sequence
  Probe
caaggctgagatccatactcagccgtctg

SEQ ID NO: 5 - DNA, 25 bp, Artificial Sequence
  Primer
aaagatcattacgatgccacctatc

SEQ ID NO: 6 - DNA, 24 bp, Artificial Sequence
  Primer
gctagcgatgaaaaacattgatca

SEQ ID NO: 7 - DNA, 22 bp, Artificial Sequence
  Probe
cagctggtccagcaccctcccc

SEQ ID NO: 8 - DNA, 24 bp, Artificial Sequence
  Primer
ttacagaaacaaaggctgtttcca

SEQ ID NO: 9 - DNA, 21 bp, Artificial Sequence
  Primer
tgagcaggcacttaacatgca

SEQ ID NO: 10 - DNA, 20 bp, Artificial Sequence
  Primer
gtacgtcttcccgagcgaaa

SEQ ID NO: 11 - DNA, 18 bp, Artificial Sequence
  Primer
ccctgtggctgatccttg

SEQ ID NO: 12 - DNA, 24 bp, Artificial Sequence
  Primer
agctagggtgtactgagaatttgg

SEQ ID NO: 13 - DNA, 20 bp, Artificial Sequence
  Primer
gtcatcgctctccacactca

SEQ ID NO: 14 - DNA, 24 bp, Artificial Sequence
  Primer
gctacatcgacatggtgtccactt

SEQ ID NO: 15 - DNA, 24 bp, Artificial Sequence
  Primer
gttgctcctacctggaaaggatga

SEQ ID NO: 16 - DNA, 24 bp, Artificial Sequence
  Primer
tgtccttctaccagggctacacaa

SEQ ID NO: 17 - DNA, 24 bp, Artificial Sequence
  Primer
tgtgctgcaaggcgattaagttgg

SEQ ID NO: 18 - DNA, 20 bp, Artificial Sequence
  Primer
cagtacacctggctgttcca

SEQ ID NO: 19 - DNA, 23 bp, Artificial Sequence
  Primer
caaacaaacagtatgatcccaga

SEQ ID NO: 20 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 21 - DNA, 33 bp, Bacteriophage P1
ataacttcgtatagcatacattaacgaagttat

(19) United States
(12) Patent Application Publication
(10) Pub. No.: US 2010/0229254 A1
(43) Pub. Date: Sep. 9, 2010

(54) METHOD FOR TARGETED CELL ABLATION

(76) Inventors: Marie KMITA, Montreal (CA); Damien Gregoire, Montreal (CA)

Correspondence Address: SMART & BIGGAR, 1000 DE LA GAUCHETIERE ST. W., SUITE 3300, MONTREAL, QC, H3B 4W5, CA

(21) Appl. No.: 12/397,612

(22) Filed: Mar. 4, 2009

(51) Int. Cl. C12N 15/01; C12N 5/06; A01H 1/02

(52) U.S. Cl. 800/21; 435/375; 800/260

(57) ABSTRACT

The present technology relates to a method for causing cell death. The method comprises the step of genetically manipulating chromosomal DNA of a cell such as by, for example, using a recombinase system, to lose an autosome during cell division and wherein loss of the autosome results in death of the cell. The technology also relates to a method for selective ablation of proliferative cells within a population of cells.

### FIELD OF INVENTION

The present technology relates to methods for causing death of targeted cells and to methods of observing the effect on a population of cells of the ablation of targeted cells within the population of cells.

### BACKGROUND

Targeted cell ablation has proven to be a valuable approach to study in vivo cell functions. Initially, cell/tissue ablations have been generated either by micro-dissection, aspiration or laser-based techniques. However, the difficulty of distinguishing neighboring but genetically distinct cell types has restricted the application of surgery-based cell ablation and has led to the emergence of alternative approaches. These latter involve antibodies targeting surface molecules, the use of chemicals that interfere with the survival of particular cell types or transgenes that will directly or indirectly trigger cell destruction.

Generally, strategies known in the art are based on the expression of a cytotoxin or a protein that renders cells sensitive to cytotoxic products. The first and most used cytotoxic product is the A chain of diphteria toxin (DT-A). DT-A triggers apoptosis through efficient inhibition of protein synthesis mediated by ADP-ribosylation of elongation factor 2. Once inside the cell, a single molecule of DT-A is sufficient to induce apoptosis (1). In this respect, DT-A is a very potent means to trigger complete ablation of cells expressing it. However, because of its drastic toxicity, it is essential that the DT-A transgene be totally silent in non-targeted cells. Any leakage in transgene expression, even at low levels, would result in non-specific cell destruction.

Other techniques are based on usage of prodrug metabolizing enzymes that are not detrimental for cell survival per se but metabolize prodrugs into cytotoxic products. For instance, the Herpes Simplex Virus thymidine kinase (HSV-tk) phosphorylates nucleoside analogs such as ganciclovir (GCV) into toxic metabolites that are inserted into replicating DNA, inhibiting replication and subsequently triggering cell death. For prodrug metabolizing enzyme, bystander effects have been reported suggesting that cell ablation could also encompass non-targeted neighboring cells as for the method based on the expression of cytotoxins.

The majority of cell ablation procedures known in the art involve exogenous cytotoxic products. It is also possible to induce cell death by triggering the endogenous apoptotic pathway. Mallet and colleagues showed that apoptosis can be induced in targeted cells by inducing the dimerization of the pro-apoptotic form of Caspase 3 (17, 2). They generated a transgenic mouse strain driving hepatocyte-specific expression of the human Caspase 3 fused to a modified FKBP domain. Subsequent injection of the FK1012 homolog, AP20187, which binds simultaneously two FKBP domains, triggered homodimerization of the FKBP-Caspase 3 fusion protein thereby inducing the Caspase 3 cell death pathway.

The particularity of inducible approaches is that they require injection of the appropriate substrate/catalyst to trigger cell death. Since cell death relies on the availability of the substrate/catalyst, cell death induction is transient. Cell ablation depends on the accessibility of the injected product to the cells. For instance, ablation of embryonic tissues cannot be achieved using DT-A injection since the toxin cannot cross the placental barrier. In addition, the injection step being a key parameter for inducible cell ablation, it is important to ensure that injections are performed with minimal variations. The HSV-tk based cell ablation technique was initially described as being restricted to proliferating cells, however it turned out that distinct non-proliferation cells were also affected. Thus none of the existing techniques of cell ablation allow for the specific death of proliferating cells versus non-proliferating cells.

Constraints regarding the procedures described above reside, at least, in the requirement of generating a specific transgene for each distinct cell population, in the use of cytotoxic products and in the lack of specificity and reliability of the procedures.

Therefore, there remains a need for more reliable, more specific and simpler methods to perform targeted cell destruction, for methods having valuable application in the study of for example, but not limited to, cellular processes, morphogenetic processes and the study of mechanisms underlying tissue/organ homeostasis and regeneration.

### SUMMARY OF THE INVENTION

According to one aspect of the present invention, there is provided a method for causing cell death, the method comprising the step of manipulating chromosomal DNA of a cell to lose an autosome upon cell division, wherein manipulation of the chromosomal DNA involves a recombination event, and wherein loss of the autosome causes death of the cell.

According to another aspect of the present invention, there is provided a method for selective ablation of targeted cells within a population of cells, wherein the targeted cells comprises a set of inverted recombinase recognition sequences in an autosome and a recombinase-encoding gene, the method comprises expressing the recombinase in the targeted cells to lose the autosome during cell division resulting in ablation of the targeted cells.

According to still another aspect of the present invention, there is provided a method of determining whether a selective ablation of targeted cells within an animal leads to a variation in the animal, the method comprising: (a) obtaining a transgenic animal having targeted cells carrying a set of inverted recombinase recognition sequences in an autosome, and carrying a recombinase-encoding gene; (b) expressing the recombinase within the targeted cells to lose the autosome during cell division, wherein loss of the autosome results in ablation of the targeted cells; and (c) determining if there is a difference between the animal having a recombinase expressed in the targeted cells and a control animal not having the recombinase expressed in the targeted cells, wherein presence of a difference indicates that ablation of the targeted cells leads to a variation in the animal.

According to a further aspect of the present invention, there is provided a method for producing a transgenic non-human organism having a targeted population of cells that have been ablated, the method comprising: (a) producing an F1 generation by crossing a first and a second transgenic parent, the first transgenic parent carrying the set of inverted recombinase recognition sequences on an autosome, the second parent carrying a recombinase-encoding; and (b) expressing the recombinase within targeted cells in an offspring of the F1 generation carrying the set of inverted recombinase recognition sequences and the recombinase-encoding gene; wherein expression of the recombinase in the targeted cells results in loss of the autosome during cell division, causing ablation of the targeted population of cells within the offspring of the F1 generation defined in (b).

These and other aspects of the invention will now become apparent to those of ordinary skill in the art upon review of the following description of embodiments of the invention in conjunction with the accompanying drawings.

### BRIEF DESCRIPTION OF THE DRAWINGS

FIG. 1 is a RNA whole-mount in-situ hybridization illustrating the expression of Cre in Prx1-Cre embryos.

FIG. 2 is a whole-mount immunodetection of the activated form of Caspase 3 showing apoptosis in invloxP/+; Prx1-Cre limb buds (right panel; arrowheads) compared to controls (left panels; arrowheads).

FIG. 3 shows a higher magnification of control and mutant forelimb buds of FIG. 2.

FIG. 4 is a TUNEL assay on cryosection of mutant forelimb bud showing apoptosis in mesenchymal cells (mes) but not in ectodermal cells (ect).

FIG. 5 is a whole-mount immunodetection of activated caspase 3 showing apoptosis in nascent mutant forelimb buds.

FIG. 6 is a whole-mount immunodetection of activated Caspase 3 showing TRIP-induced apoptosis in Cre-expressing cells of invloxP/+; Wnt1-Cre embryos (right panel; arrowhead).

FIG. 7: is a Real-time PCR detection showing loss of the loxP-carrying chromosome 2 in proliferating cells.

FIG. 8 is a graph analysing data obtained by FISH detection and showing chromosome 2 monosomy in invloxP/+; Prx1-Cre limb buds.

FIG. 9 is a graph analyzing data obtained by FISH detection and showing detection of chromosome 2 in metaphase cells from invloxP/+; Prx1-Cre forelimb buds.

FIG. 10 is an immunodetection showing the activated form of Caspase 3 on cryosections of invloxP/+ and invloxP/+; LOMP-Cre retinas.

FIG. 11 shows a scheme of wild-type, non-inverted, and inverted loxP-carrying Chr2. LoxP sites (triangles) and primers used for PCR and real-time PCR are indicated (arrows), as well as a PCR detection of DNA inversion in invloxP/+; LMOP-Cre retinas. Cre-mediated inversion of the DNA fragment flanked with loxP sites is detected with the AC and BD primer sets.

FIG. 12 is a schematic representation of an embodiment of an experimental design for TRIP-mediated ablation of proliferating cells.

It is to be expressly understood that the description and drawings are only for the purpose of illustrating certain embodiments of the invention and are an aid for understanding. They are not intended to be a definition of the limits of the invention.

### DETAILED DESCRIPTION OF EMBODIMENTS

The embodiments and implementations defined below are not intended to be exhaustive or to limit the technology to the precise forms disclosed in the following detailed description. Rather, the embodiments and implementations are chosen and described so that others skilled in the art may appreciate and understand the general principles and general practices of the present technology.

Unless defined otherwise, all technical and scientific terms used herein have the same meaning as commonly understood by one of skill in the art to which the technology pertains.

For ease of reference, the following abbreviations and designations are used throughout:

Chr: chromosome

TRIP: targeted recombination between inverted loxP sites

invloxP: inverted loxP sites, i.e., loxP sites inserted in opposite orientation

The invention stems from, but is not limited to, the discovery that targeted recombination between inverted loxP sites (TRIP) in double heterozygous embryos carrying a set of inverted loxP sites on chromosome 2 and carrying a Cre transgene expressing the Cre recombinase results in chromosome loss in proliferating cells. The invention further stems from the discovery that cells that have lost the inverted loxP-carrying chromosome were eliminated by apoptosis prior to completion of the cell cycle, which indicated that the recombination outcome was cytotoxic (16).

When loxP sites located in cis are in inverse directional orientation with respect to each other, recombination can results in the elimination of the loxP-carrying chromosome (3, 4, 5). Lewandoski and colleagues showed that combining a Y chromosome carrying loxP sites in inverted orientation with a Cre transgene expressed ubiquitously during early embryogenesis resulted in XX and XO progeny, which indicated that the Y chromosome had been eliminated (4).

The present invention provides a method for causing cell death by elimination of a chromosome in targeted proliferating cells.

In some embodiments, the method comprises the step of genetically manipulating chromosomal DNA of a proliferating cell in order to eliminate an autosome of the cell. As used herein, the expression “genetically manipulating chromosomal DNA” refers to the direct manipulation of an organism's genetic material.

In some specific embodiments, genetically manipulating chromosomal DNA of a cell involves using a recombination system. The contemplated recombination system involves a recombinase and corresponding recombinase recognition sequences. In this specific implementation, the cell targeted for cell death carries the recombinase recognition sequences as well as a recombinase-expressing gene.

The chromosome contemplated is an autosome, such as chromosome 2. According to one aspect of the present technology, loss of the chromosome results from a recombination event between the two inverted loxP sequences. Cell death results from the subsequent deleterious genomic rearrangement in proliferating cells. More specifically, cell death results from an unequal crossover between sister-chromosome giving rise to an acentric and a dicentric chromosome, eventually lost during cell division.

As used herein, the term “autosome” refers to a chromosome that is not a sex chromosome (e.g., not the X or the Y chromosomes in mouse or human genomes).

Targeted Cells

The cells contemplated by the present technology are eukaryotic cells. Examples of eukaryotic cells that are encompassed by the present technology include, but are not limited to, animal and vegetal cells. These include, but are not limited to, chordate, nematode and arthropod cells (e.g., drosophila), and more specifically to mammalian cells such as human cells, mouse cells, rat cells, etc.

The cells contemplated by the present technology are cells present in any tissue of an organism. The cells contemplated by the present technology are proliferating cells including germline, lineage-specific progenitors, stem cells, as well as differentiated cells.

Further specific examples of human or mouse cells include, but are not limited to cells of embryonic tissues (e.g., cells of the endoderm, ectoderm or mesoderm), cells of the immune system (e.g., CD4+ and CD8+ T cells, Fox3P+ T cells, B cells, dendritic cells, Langerhans cells, natural Killer cells, thymocytes, CD11c+ macrophages, mast cells), from neuronal system (e.g., myelinating glia cells, astrocytes, Schwann cells, Purkinje cells, hypothalamic AgRP+, GFAP+ Glial cells, neurons, olfactory sensory neurons, oligodendrocytes, retinal ganglion cells, neuronal stem cells, cone photoreceptors, roof plate cells), from skin (e.g., melanocytes), from intestine (e.g., intestinal stem cells, intestinal Goblet cells, Paneth cells), for the pancreas (e.g., pancreatic β-cells, pancreatic acinar cells,), etc.

Other specific, but non-limiting examples of human or mouse cells include cytokine and interleukine producing cells (e.g., IL-4 producing and IL-2 producing cells), endothelial cells, eye lens fiber cells, somatotrope cells (GH+ cells), PNMT-producing cells, thyroid follicle cells, myogenic cell lineage, cardiomyocytes, vascular smooth muscle cells, myocytes, osteocytes, osteoblasts, chondrocytes, limb mesenchymal cells, parietal cells, secretin cells, bulge epithelial, luminal cells of mammary gland, prostate notchl expressing cells, adipocytes, hepatocytes, etc.

Cells that can be subjected to the present technology are proliferating cells and such cells will be apparent to those of skill in the art.

As used herein, the expression “proliferating cells” refers to cells that grow and divide to produce daughter cells.

The present technology may be used to cause selective ablation of targeted cells within a population of cells such as ablation of targeted cells within a culture of cells, ablation of targeted cells within a tissue, an organ, a system such as, for example, the immune system, or an organism, such as, for example, an animal. The expression “selective ablation” refers to the ablation of targeted cells in preference to other cells.

As used herein, the expression “targeted cells” refers to a group of cells which may themselves be a subset of a larger population of cells. In some embodiments, the technology is applied to a population of cells comprising the targeted cells to be ablated and results in the ablation of the targeted cells specifically. The targeted cells have characteristics that distinguish them from the remaining cells in population.

Targeted cells are characterized by the expression of a gene (e.g., a recombinase-encoding gene) involved in the recombination event between two inverted recombinase recognition DNA sequences and are restricted to proliferating cells.

As used herein, the term “organism” refers to a living thing which, in at least some form, is capable of responding to stimuli, reproduction, growth or development, or maintenance of homeostasis as a stable whole (e.g., an animal, a plant, a fungus, or a micro-organism). An organism may either be unicellular or may be multicellular. A multicellular organism may be composed of many cells which may be grouped into specialized tissues or organs. The term organism when used herein to designate an animal is meant to refer to the animal at any stage of its development (e.g., the term organism may designate the animal embryo or may designate the adult animal).

Cell Death

As demonstrated herein, recombination between a set of inverted recombinase recognition sequences causes loss of the autosome and triggers death of the cell by apoptosis.

It is generally accepted that cell death can either be the consequence of a passive, degenerative process, or the consequence of an active process. The former type of cell death is termed necrosis, the latter apoptosis. Apoptosis represents the mode of death that is actively driven by the cell. On the opposite, necrosis represents a passive consequence of gross injury to the cell. Necrosis is a premature or unnatural death of cells, is morphologically different from apoptosis, and its physiological consequences are also very different from those of apoptosis. Cells which die due to necrosis do not typically send the same chemical signals to the immune system that cells undergoing apoptosis do. Apoptosis is a very common phenomenon during embryogenesis as well as adult life. The differences between the two types of death are appreciated by those of skill in the art.

Assays for the determination and quantification of apoptosis include amongst other flow cytometry assays, e.g., terminal deoxynucleotidyl transferase dUTP nick end labeling (TUNEL assay), a method for detecting DNA fragmentation by labeling the terminal end of nucleic acids. For TUNEL assay, commercially available kits can be used (e.g., Fluorescein FragEL DNA Fragmentation Detection Kit from Oncogene Research Products), Tetramethyl-rhodamine-5-dUTP (e.g., from Roche). Assays for the determination and quantification of apoptosis also include calorimetric assays, such as, for example, Caspase-3 assays. The Caspase-3 calorimetric assay is based on the hydrolysis of acetyl-Asp-Glu-Val-Asp p-nitroanilide by Caspase-3, resulting in the release of the p-nitroaniline moiety which is detected at a wavelength of 405 nm. Commercially available kits can be used (e.g., CASP3C detection kit from Sigma-Aldrich). These techniques and the way to carry them out are well known in the art.

Inverted Recombinase Recognition Sequences

As used herein, the expression “recombinase recognition sequence” refers to a nucleotide sequence which is recognized and which interacts with a recombinase and at which is catalyzed a site-specific DNA recombination event. In a specific, but non-limiting implementation of the present technology, the recombinase recognition sequence is the loxP site; a 34 base pair nucleotide sequence isolated from bacteriophage P1 (such as in Hoess et al., Proc. Natl. Acad. Sci. USA, 79:3398 (1982)). The loxP site consists of two 13 base pairs inverted repeats separated by a 8 base pairs spacer region. In a specific, but non-limiting implementation, the loxP site has a nucleic acid sequence corresponding to or homologous to the following sequence ATAACTTCGTATAGCATACATTAACGAAGTTAT (SEQ ID NO: 21). Another recombinase recognition sequence is the FRT, a distinct 34-bp Flp recombinase (from the yeast S. cerevisiae) recognition sequence.

A person skilled in the art will appreciate that variations within the nucleic acid sequence of the recombinase recognition sequence, such as for example, variation in the nucleic acid sequence of a loxP site, are permitted so long as the recombinase is capable of effecting recombination between the recombinase recognition sequences. Nucleic acid sequences homologous to the loxP sequence: ATAACTTCGTATAGCATACATTAACGAAGTTAT are also encompassed by the present technology.

The term “homology”, “homologue” or “homologous” refers to a sequence that exhibits at least 70% identity with the indicated sequence. In another embodiment, the sequence exhibits at least 72% identity with the indicated sequence. In another embodiment, the sequence exhibits at least 75% identity with the indicated sequence. In another embodiment, the sequence exhibits at least 80% identity with the indicated sequence. In another embodiment, the sequence exhibits at least 82% identity with the indicated sequence. In another embodiment, the sequence exhibits at least 85% identity with the indicated sequence. In another embodiment, the sequence exhibits at least 87% identity with the indicated sequence. In another embodiment, the sequence exhibits at least 90% identity with the indicated sequence. In another embodiment, the sequence exhibits at least 92% identity with the indicated sequence. In another embodiment, the sequence exhibits at least 95% or more identity with the indicated sequence. In another embodiment, the sequence exhibits at least 97% identity with the indicated sequence, the sequence exhibits at least 99% identity with the indicated sequence. In another embodiment, the sequence exhibits 95% to 100% identity with the indicated sequence.

Homology may be determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages, to name a few. Other suitable lox sites that may be useful in the present technology include loxB, loxL and loxR sites which are nucleotide sequences isolated from other bacteriophage strains. Other variants of the lox site also include, but are not limited to, lox511, lox571, lox2272, lox71, lox66, etc. Lox sites can also be produced by a variety of synthetic techniques which are well known in the art. Several suitable lox sites are already available commercially (e.g., Addgene, Plasmid 11584: “Lox-Stop-Lox TOPO”). The expression “a set of recombinase recognition sequences” refers to at least two recombinase recognition sequences between which recombination occurs. As used herein, the expression “inverted recombinase recognition sequences” refers to a set of recombinase recognition sequences, such as for example, a pair of loxP sites, that are in opposite directional orientation with respect to each other. Orientation refers to the general 5′ to 3′ ends of a DNA molecule.

In a specific embodiment, the set of inverted recombinase recognition sequences are located in cis (i.e., located on the same DNA molecule, such as, for example, on the same chromosome). In some further embodiments, a set of inverted recombinase recognition sequences may include three, four, five, six or more recombinase recognition sequences.

The minimum length of the DNA segment required between the set of inverted recombinase recognition sequences located in cis, is a length which allows the recombinase to perform efficient recombination between the set of inverted recombinase recognition sequences. The efficiency of recombination is however independent of the maximum length of the DNA segment between the set of inverted recombinase recognition sequences located in cis.

Recombinases

As used herein, the term “recombinase” refers to both a nucleic acid sequence encoding a recombinase protein or the recombinase protein itself. A person skilled in the art will appreciate when the term recombinase is used to designate a nucleic acid and when it is used to designate a protein. The nucleic acid sequence encoding the recombinase may be endogenous to the cell or may have been engineered into the cell such as to be stably inserted in the genome of the cell or to be carried on a vector or plasmid and placed under the control of a promoter.

By “recombinase” is intended an enzyme which catalyze DNA strand recombination. The recombinase catalyses a site specific recombination between the recombinase recognition sequences, including between a set of inverted recombinase recognition sequences.

In a specific, but non-limiting embodiment, the recombinase is a Cre recombinase encoded by the bacteriophage P1 Cre gene. As used herein, the expression “Cre gene” refers to a nucleotide sequence that encodes a gene product which effects site-specific recombination of DNA at lox sites.

The Cre recombinase sequence is that described in the art and for example in N. Sternberg et al., J. Mol. Biol, 187:197-212 (1986).

In a specific, but non-limiting implementation, the Cre recombinase has the amino acid sequence depicted in SEQ ID NO: 20.

A person skilled in the art will however appreciate that variations in amino acid sequence of the Cre recombinase are permitted without departing from the present technology.

By use of the degenerate genetic code, variant nucleotide sequences may be generated that are translated into variant recombinase, such as, for example, functional variants or functional equivalents of the Cre recombinase. Functional equivalents of the Cre recombinase may be generated by making minor sequence variations in the nucleic acid sequence encoding the Cre recombinase and measuring recombinase activity of the translated variant protein. Methods and techniques for measuring recombinase activity of a recombinase are well known in the art.

Variant proteins may also be selected with desired properties such as thermolability, thermostability, modified cellular localization, modified sequence recognition, modified frequency of recombination.

In a further embodiment, recombinases other than Cre recombinase may be used in the practice of the present technology. Other recombinases include recombinases from the tyrosine family of recombinases such as the Flp recombinase (from yeast S. cerevisiae) and its recognition sequences (FRTs) or the λ integrase from lambda phage, or a recombinase from the serine family of recombinases including, the gamma-delta resolvase (from the Tn1000 transposon), Tn3 resolvase (from the Tn3 transposon) and φC31 integrase (from the φC31 phage). Recombinases and their corresponding recombinase recognition sequences from other organisms may also be used in the practice of the present technology. In a further embodiment, Flp recombinase may be used along with the Flp recognition sequences, the FRTs as recombinase recognition sequences. Flp recombinase recognizes a distinct 34-bp minimal site. In some further embodiments, Flp is used along with inverted FRTs.

Other recombinases which mechanism involves DNA strand cleavage, exchange and ligation may also be useful in the practice of the present technology and will be recognized by skilled artisans.

Regulation of the Recombinase Gene Expression or Activity

The present technology allows for conditional expression of the recombinase. For example, the recombinase may be expressed at specific times during development, in specific cell types, in specific tissues, may be expressed during specific stages of the cell-cycle, may be expressed in the presence of specific molecule, or combination of molecules such as transcription factors and regulatory proteins, etc. The recombination event induced by the recombinase is in turn time specific, cell type specific, tissue specific, may occur during specific stages of the cell-cycle, may occur in the presence of specific molecule, etc.

In some embodiments, the recombinase-encoding gene present in the genomic DNA of a targeted cell will be naturally expressed when that portion of the genomic DNA carrying the recombinase-encoding gene is transcribed.

In other embodiments, the cell is engineered to express the recombinase under specific conditions.

As used herein, the expression “expression of the recombinase” or “expressing the recombinase” refers to the transcription of the recombinase gene and the production of an active recombinase protein.

As used herein, the expression “conditional expression” or “conditionally expressed” is intended to refer to transcription of the recombinase gene and the production of an active recombinase protein when a condition is met while no transcription of the recombinase gene and no production of an active recombinase protein is executed otherwise.

Conditional expression of the recombinase can be achieved or performed naturally by the cell (i.e., without artificial intervention) or may be achieved or performed artificially (i.e., with the involvement of artificial intervention, such as for example, but not limited to, the use of regions or promoters regulated by the use of chemical agents, etc.).

Regulation of the recombinase expression may be achieved using for example, regulatory sequences such as but not limited to, inducible promoters. Examples of inducible promoter include chemically-regulated promoters whose transcriptional activity is regulated by the presence or absence of a particular chemical agent. Inducible promoters also include promoters whose transcriptional activity is regulated by environmental factors. Tissue-specific promoter may also be used for natural conditional expression of the recombinase. As their name says, the activity of these promoters is induced by the presence or absence of biotic or abiotic factors in certain tissues of an organism. The use of these conditional regulatory sequence allows for expression of the genes linked to them to be turned on or off for example, at certain stages of development of an organism or in a particular tissue or in particular biological conditions.

As used herein, the term “promoter” refers to a nucleic acid sequence, which regulates expression of a gene associated on the same DNA molecule. Such promoters are typically known to be cis-acting sequence elements required for transcription as they serve to bind DNA dependent RNA polymerase, which transcribes sequences present downstream thereof.

There are virtually several thousands of inducible promoters that vary according to the organism source and cells or tissues where they regulate gene transcription. A person skilled in the art is familiar with the types of inducible promoters and in which conditions they are to be used.

The elements of the regulatory sequences are assembled using standard recombinant DNA techniques well known in the art. Typically, the promoter is located upstream to the DNA sequence expressing gene. The promoter is ligated in such a position and manner as to be capable of effecting the transcription of the DNA sequences into mRNA.

Conditional expression of the recombinase may be achieved by controlling transcription (e.g., promoter, enhancer, silencer, rate of elongation), post-transcriptional events (e.g., editing, splicing, message stability, polyadenylation, transport out of the nucleus), translation (e.g., initiation, elongation, termination), or post-translational events (e.g., secretion and transport, cell localization, folding, assembly, protease cleavage or degradation, acylation, glycosylation, sulfation, phosphorylation, isomerization). Transcription of the recombinase may be regulated by, for example, tetracyline, bacteriophage RNA polymerase, IPTG, heavy metal, steroid, viral infection, expression of a DNA-binding factor, modulation of a DNA-binding factor by chemical inducers of dimerization, developmental stage, heat, tissue type, or any combination thereof.

In some further embodiments, the present technology also contemplates the use of regulatory regions that are already present in the targeted cells and that function naturally. As used here, the expression “function naturally” refers to a function that exists in or that is produced by nature (i.e., without artificial intervention).

In some further embodiments, the present technology also contemplates the use of regulatory regions that function artificially (i.e., that involves artificial intervention).

Regulatory regions may be selected from any genes expressed in targeted cells, including but not restricted to genes expressed during embryonic development (e.g., Hox, Wnt-1), genes expressed in immune cells (e.g., CD4, CD8, CD11, IL2, IL4, immunoglobulin heavy chain, globin), genes expressed in neuronal cells (e.g., GFAP, Pit-1, enolase), genes expressed in muscle cells (e.g., muscle creatine kinase), genes expressed in brain cells (e.g., brain creatine kinase), genes expressed in bone marrow (e.g., elastase), genes expressed in eye cells (e.g., delta-2 crystallin gene), genes expressed under specific environmental conditions (e.g., serum-responsive genes, interferon-responsive genes, steroid-responsive genes, heat shock protein genes), etc.

In some specific, but non-limiting, embodiments, it may be useful to have ubiquitous expression of the recombinase, in these embodiments, regulatory regions may be obtained from genes such as, for example, beta-actin, phosphoglycerate kinase, HMG-CoA reductase, major histocompatibility complex class I, beta2-microglobulin, HSV thymidine kinase gene, Rous Sarcoma Virus regulatory elements, CMV immediate-early gene, SV40 origin, or the like.

In a further specific, but non-limiting embodiment, conditional expression of the recombinase is achieved in a tissue-specific manner. An example of a method to achieve tissue-specific expression of a recombinase, such as for example, the Cre recombinase, involves inserting a Cre-encoding gene in a genomic region such as the Cre expression is under the control of the Wnt-1 regulatory regions and marking these Cre-expressing cells and their descendants with a reporter of Cre activity, such as for example, Z/EG or Rosa26reporter (9, 16), to name only a few. As is appreciated in the art, Wnt-1 mRNA is detectable largely within dorsal neuroepithelium of the midgestation embryo, a tissue that gives rise to a wide variety of migratory cell population over the course of development, including the neural crest and derivatives of the rhombic lip. In this embodiment, expression of Cre is effected concomitantly with expression of Wnt-1 in the cells and tissues where Wnt-1 expression typically occurs.

In some embodiment, activation of the recombinase is regulated by a chemical agent (e.g., administration of a drug to an organism or to cells of the organism, by endogenous metabolite of the organism itself or of the cells of the organism). The chemical agent may, for example, be a ligand for a nuclear receptor. In this example, a cell may carry a recombinase that is fused to a nuclear receptor (e.g., retinoid receptor, estrogen receptor, nerve growth factor receptor, steroidogenic factor receptor, germ cell nuclear factor receptor, or the like) to give rise to a recombinase-nuclear receptor fusion protein. Introduction of a ligand specific for the nuclear receptor into such a cell triggers translocation of the recombinase fusion protein from the cytoplasm of the cell into its nucleus and subsequent expression of the recombinase.

A further example of a mechanism for achieving conditional expression of the recombinase involves the tetracycline (tet) responsive system. This system involves two components: a transactivator gene, encoding a fusion protein that specifically binds tet as well as operator sequences of the tet operon (tetO), and a tetO::recombinase transgene. The fusion protein encoded by the transactivator gene is composed of the E. coli tetracycline repressor fused to, for example, the acidic domain of the herpes simplex viral protein (VP16) transactivation domain. In the absence of tet, the fusion protein encoded by the transactivation domain binds tetO DNA to activate transcription from a minimal promoter located immediately upstream of the recombinase encoding transgene. Thus continuous tet administration is required to prevent expression of the recombinase. To induce expression of the recombinase, tet administration is suspended.

In some further embodiments, the recombinase-encoding gene is under the control or more than one type of regulatory regions. In these embodiments, the recombinase is expressed under different conditions.

Selective Ablation of Targeted Cells

In a further embodiment, the present technology provides for a method for the selective ablation of targeted cells within a population of cells. In this specific embodiment, the population of cells comprises a set of inverted recombinase recognition sequences inserted in a chromosome, preferably in an autosome, such as chromosome 2, and a recombinase-encoding gene which is under conditional expression. The targeted cells are cells in the population which combine proliferation and the expression the recombinase. The recombinase-encoding gene may be stably inserted into the genome of the targeted cells or may be on a plasmid or vector within the targeted cells.

In another specific implementation of this embodiment, the method also comprises effecting expression of the recombinase within the targeted cells to cause recombination between the inverted recombinase recognition sequences. The recombination event results in loss of the autosome during cell cycle and causes death of the targeted cells, whereas other cells of the population, in which there was no expression of the recombinase or in which there was no proliferation are not affected.

In a further embodiment, the present technology provides for a system for selective ablation of targeted cells. The system comprises a recombination system such as a Cre-invloxP recombination system. When the system is present in proliferating cells and the conditions are met so that the recombinase is expressed, recombination between the set of inverted recombinase recognition sequences takes place and results in the loss of the chromosome carrying the invloxP sites and results in death of the proliferating cell.

In a further specific embodiment of the present technology, the cells targeted for ablation are heterozygous for the set of inverted recombinase recognition sequences and recombination between the set of inverted recombinase recognition sequences results in loss of the chromosome carrying the set of inverted recombinase recognition sequences. In another embodiment, the targeted cells are homozygous for the set of inverted recombinase recognition sequences and recombination between these sequences results in loss of the homologous chromosomes carrying the set of inverted recombinase recognition sequences. A person skilled in the art will appreciate that the locus of insertion of the set of the inverted recombinase recognition sequences on the chromosome is not critical.

The location of the recombinase-encoding gene is independent from the location of the inverted recombinase recognition sequences and, for thus, could be located or not on the same chromosome.

In a further specific, but non-limiting embodiment, the recombinase-mediated recombination between the set of inverted recombination sequences takes place in a proliferating cell, such as during the S or G2 phase of the cell cycle, results in the loss of the chromosome carrying the set of inverted recombinase recognition sequences and causes death of the cell.

Cell Transformation

In a specific embodiment of the present invention, recombinase recognition sequences such as for example, loxP sites in inverted orientation or the recombinase coding gene are transiently inserted into a host cell. In a specific embodiment of the present invention, recombinase recognition sequences such as for example, loxP sites in inverted orientation or the recombinase coding gene are stably inserted into chromosomal DNA of a host cell. In another embodiment, the cells carrying a set of recombinase recognition sequences also carry a recombinase-encoding gene. Such cell may be engineered to express a recombinase.

Techniques for introduction of an exogenous nucleic acid sequence into genomic DNA of a host cell are well known in the art. These methods typically include the use of a DNA vector to introduce the sequence into the DNA of a cell.

As used herein, the expression “stably inserting a sequence into a genome” or “stable insertion” or “stable incorporation” is intended to refer to insertion in a manner that results in inheritance of such sequence in copies of such genome.

As used herein, the term “vector” includes plasmids and viruses. In some embodiments, the exogenous nucleic acid sequence is introduced by any transforming means such as electroporation or transfection. The methods of cell transformation are well known to those of skill in the art.

A person skilled in the art is familiar with the techniques for integrating a transgene into a host genome and the way of carrying out these techniques.

Animals Carrying Transgenes

The expression “transgenic animal” is intended to refer to an animal which has incorporated a sequence of DNA (e.g., inverted recombinase recognition sequence) or a transgene (e.g., a recombinase linked to regulatory sequences). Because the DNA or the transgene is incorporated in all tissues including in the germ line tissue, it is passed from the parent to the offspring establishing strains of transgenic animal from a first founder animal in a mendelian manner. In a more specific embodiment, the transgenic animal is a mammal. In a further more specific embodiment, the transgenic mammal is, but not limited to, a transgenic mouse or a transgenic rat. Alternatively, the transgenic animal is, in some embodiments, a transgenic non-mammal, such as a transgenic plant or a transgenic insect (e.g., transgenic drosophila).

In some embodiments, the transgenic animals of the present technology are produced by introducing transgenes into the genetic material of the animal. Methods used to introduce a transgene in an animal include, but are not limited to, microinjection of zygotes, transformation of embryonic stem cells and retroviral integration.

With the method of microinjection, a zygote is a target cell for microinjection of transgenic DNA sequences. The use of a zygote for introduction of transgenes has the advantage that, in most cases, the injected transgenic DNA sequences will be incorporated into the host genome before the first cell division. As a consequence, all cells of the resultant transgenic animals stably carry an incorporated transgene.

In some further embodiments, embryonic stem cells may serve as target cells for introduction of transgenes of the invention into animals. Embryonic stem cells may be obtained from pre-implantation embryos that are cultured in vitro. Embryonic stem cells that have been transformed with a transgene can be injected in an animal blastocyst, after which the embryonic stem cells colonize the embryo and contribute to the germline of the resulting transgenic animal.

Retroviral infection may also be used to introduce a transgene into an animal. In some embodiments, retroviral infection can be used to introduce DNA (e.g., inverted recombinase recognition sequence) or a transgene at different stages of an animal's development. For example, retroviral infection may be used to introduce a transgene in the cells of a developing embryo or in the adult animal. Retroviral infection may also be used to introduce a transgene ex vivo in cells of a culture. In the situation where retroviral infection is used to introduce a transgene into a developing embryo, the embryo can be cultured in vitro to the blastocyst stage. During this time, the blastomeres can be targets for retroviral infection. Efficient infection of the blastomeres is obtained by enzymatic treatment to remove the zona pellucida. The retroviral vector system used to introduce the transgene is typically modified so as to be replication-defective and to carry the transgene. Transfection is easily and efficiently obtained by culturing the blastomeres on a monolayer of retrovirus-producing cells. Alternatively, infection can be performed at a later stage. Retrovirus or retrovirus-producing cells can be injected into the blastocoels. In addition, it is also possible to introduce a transgene into the germline, albeit with low efficiency, by, for example, intrauterine retroviral infection of the midgestation embryos.

In specific embodiments, retroviral infection can be used to introduce into the cell both DNA for inverted recombinase recognition sequences and a recombinase transgene.

Breeding of Animals Carrying Transgene

Another specific example of a system encompassed by the present technology is a system with a first strain of modified non-human animal carrying a set of inverted loxP sites, and a second strain of transgenic non-human animal carrying a Cre recombinase transgene.

By breeding these two strains, the Cre recombinase transgene and its substrate will be present in some of the F1 offspring. The present technology also provides a method for producing a transgenic animal having the system such as a Cre-invloxP recombination system. The method comprises producing an F1 generation by crossing a first and second transgenic parent, a first parent carrying in its genome a set of inverted recombinase recognition sequences and a transgene (“the indicator”) the expression of which serve to indicate that a recombination event has occurred and provides a permanent record of this event by transforming it into a heritable lineage marker; and a second transgenic parent harboring a recombinase gene. The elements comprised within an indicator transgene include, but are not limited to, a reporter gene, functionally silenced by, for example, insertion of a STOP cassette that has been flanked by recombinase recognition sites; and a ubiquitous active promoter capable of driving reporter expression in all cell types and at all stages of development, such that following a recombination event in any given cell, that cell and its progeny will be marked regardless of subsequent fate specification. The genome of the second parent carries a recombinase-encoding gene under conditional expression.

The expression “offspring” refers to any product of the mating of the genetically modified animal carrying an inverted recombinase recognition sequences. This term also includes any germ cell of the transgenic animal which can be used to propagate a further animal comprising the transgenes encompassed by the present technology.

An example of a method useful for identifying the offspring carrying the set of inverted recombinase recognition sequences and the recombinase-encoding gene, comprises obtaining a tissue sample from the animal offspring, such as for example, from an extremity of an animal (e.g., a tail) and analyzing the sample for the presence of nucleic acid sequences corresponding to the DNA sequence of a unique portion or portions of the transgene. The presence of this nucleic acid sequence may be determined by, for example, but not limited to, Southern blot hybridization analysis, analysis of the products of PCR reactions using DNA sequences in a sample as substrates, oligonucleotides derived from the transgene's DNA sequence, and the like.

In another embodiment, the method further comprises selecting among the offspring of the F1 generation for transgenic animals which carry the set of inverted recombinase recognition sequences and the recombinase-encoding gene. Expression of the recombinase produces in targeted cells a recombination event between the set of inverted recombinase recognition sequences which results in loss of the chromosome carrying the set of inverted recombinase recognition sequences and further results in death upon proliferation.

It is to be understood that targeted cell death will not occur in cells of the offspring thereof that do not carry both the inverted recombinase recognition sequences.

In one specific embodiment, the method further comprises the analysis of the embryos from the F1 offspring containing both Cre recombinase transgene and a set of the inverted recombinase recognition.

In some embodiment, the method further comprises the analysis of the adults from the F1 offspring containing both Cre recombinase transgene and a set of the inverted recombinase recognition.

Any Cre-containing mouse strains, created, for example, for the purpose of gene deletion analyses, such as in U.S. Pat. No. 4,959,317, could be suitable to generate an offspring containing both Cre recombinase transgene and a set the inverted recombinase recognition, such as Cre-invloxP system.

Animals may also be produced that have more than one distinct population of targeted cells. In this specific embodiment, the offspring (the F1) as defined above, carrying in targeted cells the set of inverted recombinase recognition sequences and the recombinase-encoding gene under a particular conditional expression (e.g., promoter inducible in GFAP-expressing neurons) is breed with a further transgenic strain carrying the recombinase-encoding gene under a different type of conditional expression (e.g., promoter inducible in CD4-expressing T cells). In the offspring (the F2), Cre-mediated recombination and cell death may occur in cells of both populations (e.g., in GFAP-expressing neurons and CD4-expressing T cells), causing their death upon proliferation.

In a specific implementation of this embodiment, the recombinase is naturally expressed such as during the development of the embryos or in the adult.

In a further embodiment of the present technology, there is provided a transgenic non-human animal in which a targeted population of cells has been ablated. The animal may be an adult or an embryo. The targeted cells comprise a set of inverted recombinase recognition sequences inserted in an autosome and comprise a recombinase-encoding gene under conditional expression. The cells have been ablated from the loss of the autosome carrying the set of inverted recombinase recognition sequences resulting from expression of the recombinase and recombination between the set of inverted recombinase recognitions sequences.

Characterization of Animals Harboring Cell Ablation

In a further embodiment, the present technology provides for a method of observing the effect of selective death of targeted cells within a population of cells within an animal. The method comprises obtaining a transgenic animal having a set of inverted recombinase recognition sequences inserted in a chromosome, preferably an autosome, most preferably in chromosome 2, and a recombinase-encoding gene under conditional expression wherein targeted cells within the population of cells, are proliferating cells expressing the recombinase. The recombinase within the targeted cells effect recombination between the set of inverted recombinase recognition sequences and induces loss of the autosome then causing death of the proliferating cells. The method also comprises the step of comparing the population of cells from a transgenic animal in which the recombinase is present with the one from a transgenic animal in which the recombinase is absent.

In a further embodiment, the present technology provides for a method of observing the effect of selective death of targeted cells within a population of cells within an animal. The method comprises obtaining a transgenic animal having targeted cells comprising a set of inverted recombinase recognition sequences inserted in a chromosome, preferably an autosome, most preferably in chromosome 2, and comprising a recombinase-encoding gene under conditional expression. The method comprises activating the recombinase within the targeted cells to effect recombination between the set of inverted recombinase recognition sequences to induce the loss of the autosome and to cause death of the proliferating target cells. The method also comprises the step of comparing a group of animals in which the recombinase has been activated in the targeted cells with a group of animals in which the recombinase has not been activated in the targeted cells.

In any cases, comparing the transgenic animal in which the recombinase has been expressed in the targeted cells with a control animal may be done by following or monitoring one or more properties of the animal or of the population of cells comprising the targeted cells using techniques such as, but not limited to, microscopy, immunodetection, cell counts, chromosome quantification, fluorescent in situ hybridization, which are well known in the art, or by following or monitoring the phenotype of the animal.

This comparison allows determining whether ablation of the targeted cells has an effect on the population of cells and/or on the animal and may further reveal what impact this ablation has on the animal.

In a further embodiment, the present technology provides for a method of determining whether selective ablation of targeted cells within an animal leads to any change or any variation in the animal. The method involves obtaining an animal having targeted cells carrying a set of inverted recombinase recognition sequences in a chromosome, preferably an autosome, most preferably chromosome 2, and carrying a recombinase-encoding gene under conditional expression using the techniques as defined above. Expression of the recombinase within the targeted cells effects recombination between the set of inverted recombinase recognition sequences causing loss of the autosome and death of the targeted cells. The method comprises detecting or determining if a difference or a variation exists or arises between the animal in which the recombinase has been expressed in the targeted cells and a control animal.

Examples of differences or variations that may be detected include, but are not limited to, differences in the phenotype and fertility of the animal, in its behavior (e.g., learning, memory, aggressiveness), in biological processes of the animal including organ physiology and ability to regeneration, in its development (e.g., development of tissues, organs, systems, etc.), etc. Differences may also be detected at the cellular level, for example, difference may be observed in the biochemical properties of the cells, in the phenotype of the cells, in the differentiation of the cell population, etc. The presence of a difference indicates that ablation of the targeted cells leads to changes in the animal. For example, this method may be used to observe the effect of destruction of a targeted cell population on development processes such as morphogenetic processes and mechanisms underlying tissue/organ developments, gene imprinting, cell growth, cell fate determination, cell differentiation, cell death, embryology, immunological processes, body patterning, tissue repair and tissue generation, and vascularisation.

The present technology is also useful for development of animal models for, inter alia, the study of developmental processes. Ablation of a particular cell population within a developing embryo using the technology defined herein, may be used to determine the implication of this particular cell type in the development of the animal and evaluate the impact of disappearance of this cell population on, for example, development of a tissue, an organ, or a system within the animal.

In further specific, but non-limiting embodiments, the present technology is used to introduce variation in the immune system (i.e., to reduce the activation or efficacy of the immune system). In such specific embodiments, the present technology is used to cause death of targeted proliferating cells of the immune system by using the Cre-invloxP system (e.g., ablation of proliferating CD4 positive cells by using a CD4 promoter for driving the expression of Cre in a transgenic animal).

In some other embodiments, the present technology is used for the in vivo study of cell functions during organogenesis, tissue homeostasis and/or regeneration. The present technology may also be used for confirming the role of precursor cells in cell differentiation.

For example, the technology may be used in studies of brain repair and neurodegenerative disorders. More specifically, the technology may be used to study how neural progenitor stem cells introduced in animal models proliferate and differentiate into specific phenotypes, and to study how these cells migrate to and/or integrate into existing neural and synaptic circuits. Proliferation and differentiation of neuronal progenitor cells may be controlled by use of the present technology, wherein in such application, the neural progenitor cells may be ablated at specific time of their differentiation or in specific tissues.

In another specific example, animals may be produced having a targeted population of cells within the immune system carrying a set of inverted recombinase recognition sequences and a recombinase-encoding gene. The recombinase induces a recombination event in these cells leading to depletion of these cells. This may be useful in studying regeneration of the immune system.

The present technology represents a novel way of creating clinically relevant animal models of human disease. For many degenerative disorders, such as hepatitis, the TRIP technology is used to create transgenic animals with controlled degrees of liver failure that model the structure and functional deterioration that occurs in human hepatitis. These animals will allow researchers to study potential new therapies. The same approach could also be applied to the development of animal models of other diseases characterized by cell death, such as type 1 diabetes (pancreatic islet cells) and congestive heart failure (myocardial cells).

In specific embodiments, the technology may be used to develop and/or test therapeutic agents. In this embodiment, an animal model may be produced having a targeted cell population ablated (e.g., an animal having specific cells of the nervous system or cells of the vascular system ablated) such as to reproduce a disorder or a condition for which treatment is sought. Different potential therapeutic agents may be administered to such an animal model in order to assess the efficacy and/or specificity of the therapeutic agents on the treatment of the disorder or condition.

As such, the present technology may also be used in vivo or in vitro to modify or direct cellular and organ development as well as to control progression of development by selectively ablation of targeted cells of a developing tissue or organ or by ablating cells having a specific cell fate.

In some other embodiments, the present technology is used for eliminating cells that display uncontrolled growth, that have become invasive or cells that have lost or changed their differentiated properties, i.e., cells that have acquired properties that such a cell would not normally acquire during its life-span (e.g., cancer cells). The present technology may also be use to control proliferation of specific cell types.

Experiments and Data Analysis

### EXAMPLE 1

Recombination Between loxP Sites with Inverse Orientation Induces Apoptosis

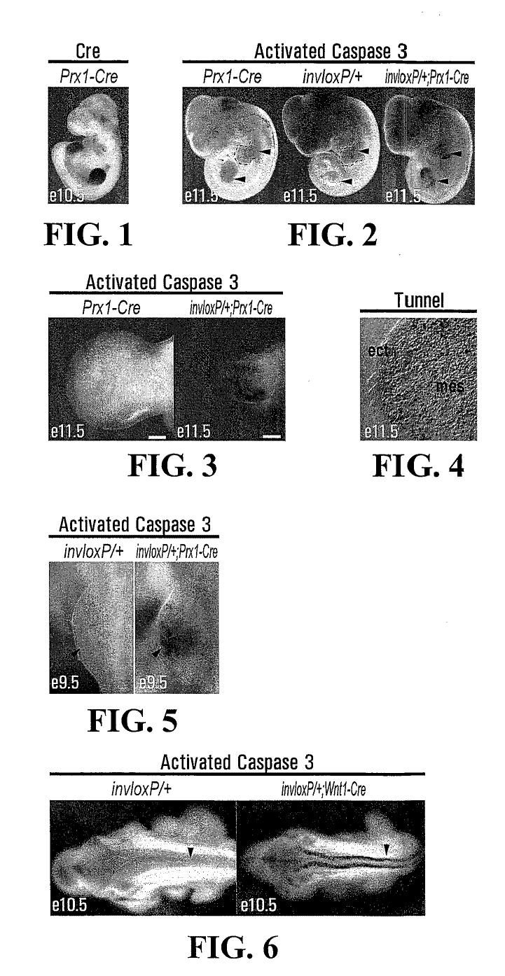

Recombination between loxP sites in inverted orientation has been proposed as a tool to induce a targeted loss of chromosome and monosomies in a tissue-specific manner (4), which would circumvent the embryonic lethality associated with constitutive autosomal monosomies (6). This approach is used herein to generate a tissue-specific monosomy of chromosome 2 (Chr2). A mice having a set of loxP sites in inverted orientation within the 5′ part of the HoxD gene cluster (referred to as invloxP hereafter) (7) was crossed to a mice carrying a Cre transgene expressed primarily in developing limbs (Prx1-Cre) (FIG. 1 and Ref. 6). It was found that limb buds of invloxP/+; Prx1-Cre embryos were severely reduced in size as compared to wild type ones. In contrast, embryos carrying only the invloxP allele or the Prx1-Cre transgene were indistinguishable from wild type embryos (FIGS. 2 and 3) and were used as controls in subsequent analyses.

Immunodetection of the activated form of Caspase 3 and TUNEL assays revealed that there was massive apoptosis in the mesenchyme of invloxP/+; Prx1-Cre limb buds (FIGS. 2, 3 and 4). In contrast, adjacent ectodermal cells, which did not express the Cre recombinase were unaffected (FIG. 4), which established that the induced ectopic cell death was restricted to Prx1-Cre expressing cells. Apoptosis was detected already in nascent limb buds indicating that ectopic cell death began soon after the expression of the Cre recombinase (FIG. 5).

### EXAMPLE 2

TRIP Results in the Depletion of Genetically Defined Cell Populations

The present analyses revealed that TRIP resulted in widespread cell death within the Cre-expression domain. To assess the severity of this effect, the Z/EG reporter transgene, permanently expressing the Green Fluorescent Protein (GFP) in Cre-expressing cells and all their progeny (9) was used to label the Prx1-Cre lineage in both control (Z/EG; Prx1-Cre) and mutant (invloxP/+; Z/EG; Prx1-Cre) limb buds. Limb buds were dissected out from control and mutant embryos at 49-50 somite-stage and the total number of cells expressing the GFP was determined. Between 1,000 and 13,000 GFP positive cells remained per mutant limb bud while control buds contained about 470,000 GFP positive cells (data not shown) indicating that 48 hours after the onset of Cre expression less than three percent of the Prx1-Cre cell lineage remained.

In this specific, but non-limiting example, the DNA fragment flanked by the two loxP sites contained two genes, Hoxd12 and Hoxd11. Limb buds of heterozygous embryos carrying either the inversion (7) or deletion (10) of this piece of DNA were morphologically indistinguishable from wild type buds. Therefore, it was concluded that the ectopic apoptosis induced following Cre-mediated recombination was not due to impaired function of HoxD genes. To further confirm that cell death was unrelated to the function of HoxD genes, the effect of combining the invloxP allele with the Wnt1-Cre transgene, which is expressed in neural crest cells (11) was investigated. Analysis of invloxP/+; Wnt1-Cre embryos showed massive apoptosis in the entire Wnt1-Cre expression domains (FIG. 6) including in embryonic regions that never expressed 5′ HoxD genes. Here again, embryos carrying only the Cre transgene or the inverted loxP sites were indistinguishable from wild type embryos (FIG. 6 and data not shown).

These results indicate that Cre-mediated recombination between loxP sites in opposite orientation triggered apoptosis and resulted in the specific depletion of Cre-expressing cells.

### EXAMPLE 3

Loss of the loxP-Carrying Chromosome Affects Cell Survival

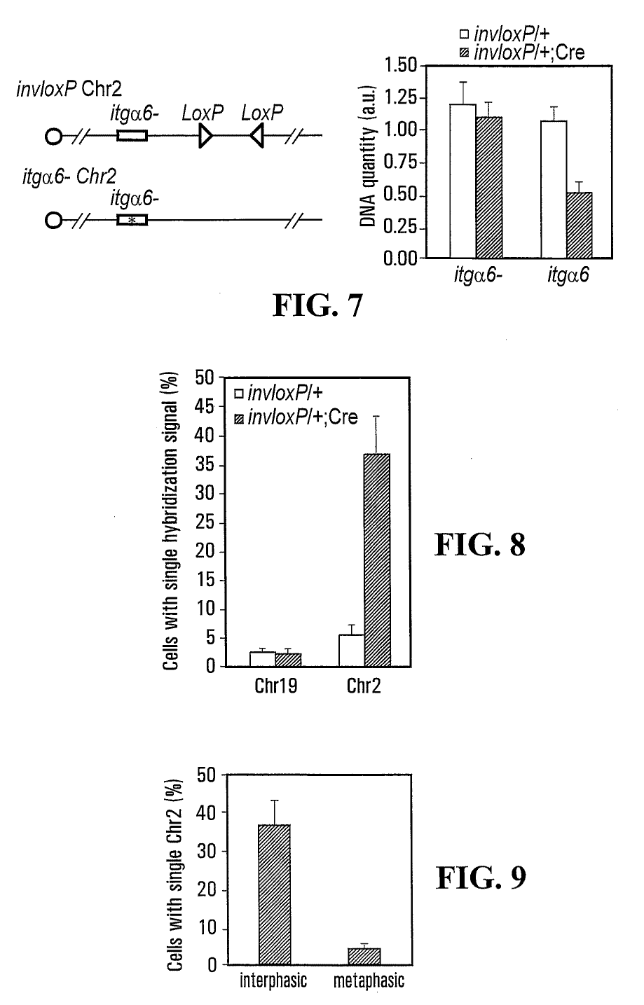

Because recombination between cis-located loxP sites in inverse orientation could result in the elimination of the loxP-carrying chromosome (1, 3), it was investigated whether the induction of ectopic cell death was associated with chromosome loss. For this purpose, a chromosome quantification in limb bud cells was first performed. In order to distinguish the loxP-carrying Chr2 from its homologous counterpart, embryos were produced with one copy of Chr2 carrying the invloxP allele and a wild-type allele of integrin alpha 6 (Itgα6) and the other copy of Chr2 carrying a mutant allele of Itgα6 (12), with or without the Cre transgene (FIG. 7). Quantification by real-time PCR of the wild type versus mutant Itgα6 allele provided a means to establish the relative proportion of the loxP-carrying Chr2 versus its homologue. It was found that the wild type Itgα6 allele was almost 2-fold under-represented in invloxP/Itgα6−; Prx1-Cre limb bud cells as compared to invloxP/Itgα6− (FIG. 7, right panel), indicating that, in invloxP/+; Prx1-Cre embryos, the Chr2 carrying inverted loxPs was missing in about half of the limb bud cells at each given time.

To independently assay for chromosome loss, a fluorescent in situ hybridization (FISH) was performed for Chr2 on dissociated cells from invloxP/+ (control) and invloxP/+: Prx1-Cre (mutant) forelimb buds. FISH for Chr19 was used as internal control. Among live cells isolated from mutant buds, 37% (±5%) had a single hybridization signal with Chr2-specific probes (FIG. 8). In marked contrast, we repeatedly detected both copies of Chr2 in metaphasic cells (FIG. 9). This result indicated that cells that have lost the loxP-carrying chromosome have been eliminated by apoptosis before entering in metaphase. The striking absence of cells with a Chr2 monosomy in M phase in contrast to the high incidence of monosomy in interphase (over one-third of cells contained a single Chr2) indicated that cell death had occurred as a consequence of chromosome elimination.

### EXAMPLE 4

TRIP Mediated Cell Death of Proliferating Cells

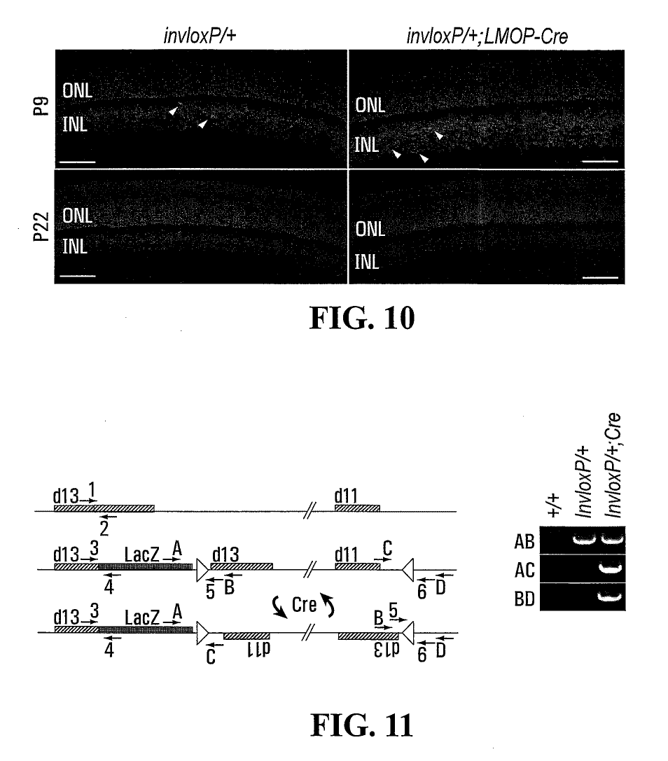

The elimination of a chromosome as a consequence of Cre-mediated recombination between inverted loxP sites was proposed to be the result of unequal crossover between sister chromatids, after DNA replication and prior to entry into anaphase (4). The observations presented herein indicate that cells that have lost one copy of their Chr2 were eliminated before completion of the cell cycle indicate that TRIP-mediated cell death was associated with chromosome loss. To test whether apoptosis was induced in proliferating cells, the effects of TRIP on post-mitotic cells were examined. Mutants carrying the invloxP allele together with the LMOP-Cre transgene, which is specifically expressed in post-mitotic photoreceptor cells (13) were generated. Retinas of invloxP/+; LMOP-Cre mice were indistinguishable from controls. TUNEL assay and immunodetection of the activated form of Caspase 3 on retinas of 9 days (P9) and 22 days (P22) invloxP/+; LMOP-Cre and control mice did not reveal any ectopic cell death in mutant retinas (FIG. 10), suggesting that Cre activity in post-mitotic photoreceptor did not induce cell death. Nevertheless, inversion of the DNA fragment located in between both inverted loxP sites (FIG. 11) was detected, indicating that recombination had occurred but did not affect cell survival. A possibility existed; however, that Cre-mediated recombination in post-mitotic photoreceptor had triggered cell death but was barely detectable due to ineffective Cre-mediated recombination. Although this latter possibility was unlikely based on the characterization of the LMOP-Cre transgene previously reported (13), the efficiency of LMOP-Cre mediated recombination was verified. For this purpose, the cell population carrying the inversion of the DNA fragment flanked with loxP sites was quantified. The inversion event being reversible due to the maintenance of the two loxP sites following recombination, an average of 50% of Cre expressing cells was expected to carry the inverted DNA fragment at a given time point. Quantification of the inverted fragment showed that, while only 14% of photoreceptors carried the inverted allele at P9 (instead of the 50% expected), the inverted DNA fragment was present in 48% of the photoreceptor population at P22 (data not shown). These results indicated that LMOP-Cre triggered recombination only in a quarter of photoreceptors at early stages of Cre expression but recombination extended to almost the entire population by P22. Nevertheless, ectopic cell death was not observed at these stages indicating that TRIP did not trigger apoptosis in post-mitotic cells. Furthermore, there was no chromosome loss detectable (data not shown), which confirmed the link between chromosome loss and the induction of apoptosis.

FIGS. 1 to 6 show that targeted recombination between inverted loxP sites (TRIP) induces apoptosis. More specifically, in FIG. 1, Cre mRNA is detected by whole-mount in Situ Hybridization in e10.5 Prx1-Cre embryo. In FIG. 2, whole-mount immunodetection of the activated form of Caspase 3 shows massive apoptosis detected in invloxP/+; Prx1-Cre limb buds (right panel; arrowheads) but not in controls (left panels; arrowheads). FIG. 3 is a higher magnification of control and mutant forelimb buds at e11.5. FIG. 4 is a TUNEL assay on cryosection of mutant forelimb bud. Induced apoptosis is detected in mesenchymal cells but not in ectodermal cells. FIG. 5 is a whole-mount immunodetection of activated caspase 3 in nascent mutant forelimb buds (e9.5). FIG. 6 shows that TRIP-induced apoptosis is detected in Cre-expressing cells of invloxP/+; Wnt1-Cre embryos (right panel; arrowhead) mes: mesenchyme, ect: ectoderm invloxP: Chromosome 2 carrying inverted loxP sites. Scale bar, 200 μm.

FIGS. 7 to 9 show that TRIP results in chromosome loss and cell death in proliferating cells. FIG. 7 shows a Real-time PCR detection of loss of the loxP-carrying Chr2. Chr 2 carrying inverted loxP sites (triangles) was combined with a Chr2 with a mutation in the integrin α6 gene (Itgα6-) Integrin α6 quantification values were normalized using the Hoxa13 gene located on Chr6. Data are presented as means±range from two independent experiments. FIG. 8 shows a FISH detection of Chr2 monosomy. In e10.5 invloxP/+; Prx1-Cre limb buds, some cells showed two hybridization signals for Chr2 (left panel, top row) while others had a single hybridization signal (left panel, middle and bottom rows). For each experiment, at least 200 cells for each genotype were analyzed. Data are presented as means± SD from three independent experiments. Background level of cells with single signal for Chr19 or Chr2 (3-6%) most likely corresponds to technical limitation or FISH signals in close vicinity to each other. FIG. 9 shows a FISH detection of Chr2 in metaphase cells from invloxP/+; Prx1-Cre forelimb buds at e10.5. More than 50 metaphases were analyzed. A representative metaphase showing two copies of Chr2 (arrowheads) is shown. Data are presented as means±SD from three independent experiments.

FIGS. 10 and 11 show that TRIP mediated chromosome loss and cell death is specific to proliferating cells. FIG. 10 shows immunodetection of the activated form of Caspase 3 on cryosections of invloxP/+ and invloxP/+;LMOP-Cre retinas at P9 and P22. Nuclei were counterstained with DAPI. Some apoptotic cells (arrowheads) were detected in the Inner Nuclear Layer (INL) at P9. No difference was detected between control and mutant retinas. FIG. 11 shows a scheme of wild-type, non-inverted, and inverted loxP-carrying Chr2. LoxP sites (triangles) and primers used for PCR and real-time PCR (arrows) are indicated. FIG. 11 also shows aPCR detection of DNA inversion in invloxP/+;LMOP-Cre retinas at P22. Cre-mediated inversion of the DNA fragment flanked with loxP sites is detected with the AC and BD primer sets.

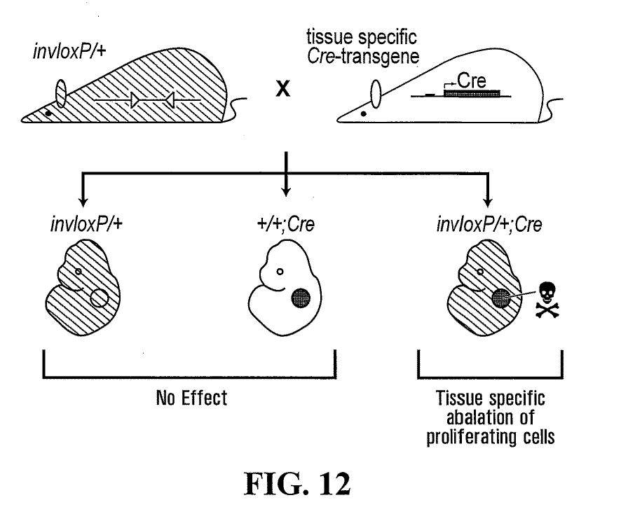

FIG. 12 illustrates a specific, but non-limiting embodiment of a design for TRIP-mediated ablation of cells, particularly proliferating cells. The mouse strain carrying the chromosome 2 with loxP sites in inverse orientation (triangles, invloxP allele) is crossed with a mouse expressing a Cre transgene under the control of a tissue-specific promoter. In the resulting progeny, ablation of proliferating cells due to the recombination between the inverted loxP sites occurs in Cre-expressing tissue (shaded) of double heterozygous specimens (invloxP/+; Cre).

Material and Technical Protocols

Animals—Mouse lines used in this work were described previously: InvloxP (7), Prx1-Cre (8), Wnt1-Cre (11), LMOP-Cre (13), Z/EG (9). Genotyping was done by southern blot analysis using genomic DNA isolated from tail biopsies or yolk sac.

Briefly, for the InvloxP mouse line, the inversion was engineered with two loxP sites in cis with opposite orientations. The first site was inserted between Hoxd10 and Hoxd11, whereas the other was inserted within Hoxd13, along with lacZ reporter sequences.For the Prx1-Cre mouse line, briefly, the Cre recombinase gene was placed under the regulation of the Prx1-derived regulatory element. An insulator element from the chicken β-globin domain (5′HS4) was placed at the 5′ end of the transgenic construct to protect against position effects at the site of transgene integration. This construct was microinjected into the pronuclei of fertilized C57BL/6J X SJL/J F2 hybrid zygotes. Transgenic founder animals were identified from the resulting litters by PCR analysis of tail DNA. Crossing to wild-type animals revealed one line passing the Prx1-Cre transgene to its offspring. To identify if this line expressed Cre at a high level, in an appropriate manner and to determine the efficiency of recombination in those embryos, Prx1-Cre mice were crossed to the Z/AP reporter line in which the histochemical marker human placental alkaline phosphatase is transcriptionally activated following Cre-mediated recombination (8).

Apoptosis detection—Whole-mount immunodetection of cleaved Caspase 3—Embryos were treated with 5% H₂O₂ in methanol for 1 hour, blocked in PBSMT (1×PBS, 2% milk, 2.5% Triton X-100) for 1 hour, and incubated with anti-Caspase 3 antibody (Cell signaling, #9661) 1:100 in PBSMT, overnight at 4° C. After extensive washes for 5 hours in PBSMT, embryos were incubated with AP-conjugated goat anti-rabbit (Santacruz) 1:2000 in PBSMT overnight at 4° C. Embryos were equilibrated in NTMT (100 mM Tris pH 9.5, 100 mM NaCl, 50 mM MgCl2, 0.1% Tween-20) and alkaline phosphatase activity detected using NBT/BCIP substrate (Roche).

Apoptosis detection on cryosections—Apoptotic cells were detected by immunodetection of cleaved Caspase 3 (Cell signaling, #9661) or the deoxynucleotidyltransferase-mediated dUTP-biotin nick-end labelling assay (TUNEL, Promega) on cryosections of limb buds or retinas (14 μm) following classical procedures and manufacturer's instructions.

Cell counts—InvloxP and Prx1-Cre mice strains were combined with the reporter strain Z/EG to obtain invloxP/+; Prx1-Cre; Z/EG and Prx1-Cre; Z/EG embryos. Forelimbs buds were dissected and separately submitted to collagenase treatment (500 U/mL, 60 min at 37° C.) to dissociate cells. GFP positive cells were counted using a Bright-Line® hematocytometer (Reichert) observed with a GFP filter on a Leica DM6000B.

Chromosome Quantification—The integrin α6 (Itgα6) locus was used as a marker to quantify each chromosome 2 by real-time PCR. The wild-type Itgα6 allele was located in cis to the inverted loxPs while a mutated integrin α6 allele (itgα6−) (12) was on the other chromosome. Real-time PCR was performed using TaqMan® probes and primers specific for mutated and wild-type Itgα6. DNA was purified from six forelimb buds isolated from, respectively, invloxP/Itgα6− and invloxP/Itgα6−; Prx1-Cre embryos at e10.5. Taqman® real-time PCR was carried out according to the manufacturer's protocol (Applied Biosystems). Hoxa13 (Chr6) quantification was used as reference for normalization.

Fluorescent In Situ Hybridization—Cells from dissected e10.5 forelimb buds were dissociated using collagenase, and treated following classical procedures to obtain interphasic and mitotic chromosome preparations (2). In situ hybridization was performed following standard protocol (14). BACs used as template for probe synthesis were RP24-63014 and RP23-463J10 for chromosome 2 (used separately in independent experiments), and RP23-125F3 for chromosome 19. Biotin and digoxigenin probes were generated by nick translation (Roche), following the manufacturer's instructions, and detected with streptavidin-alexa 546 (Molecular Probes) and anti-DIG antibody (Roche), respectively. Analysis of Chr2-specific hybridization signals was restricted to cells that accurately hybridized with Chr19-specific probe. Two hybridization dots in close vicinity to each other, likely corresponding to replicating loci, were scored as one signal.

Detection and quantification of DNA inversion—InvloxP/+ and invloxP/+; LMOP-Cre eyes were collected from P9 and P22 animals. Genomic DNA was extracted, then purified using QIAquick Kit (Qiagen). We determined DNA concentration accurately using Nanodrop 1000 (Thermo scientific) and diluted DNA to 10 ng/mL in Tris 10 mM pH8.0, 1 mg/mL RNaseA. Quantitative real-time PCR analyses were carried out with Quantitect SYBR Green PCR Kit (Qiagen) on a Mx3000P cycler (Stratagene) following manufacturer's instructions. Standard curves for quantification were generated from dilution series of genomic DNA or purified “BD” PCR fragment. Primers pair encompassing wild-type Hoxd13 locus was used as a reference for normalization. Photoreceptors correspond to 72% of the total cell population of the retina (15). Therefore raw data were divided by 0.72 to obtain the actual percentage of inverted allele within the photoreceptor cell population.

Primer and Probes Sequences for Chromosome Quantification (FIG. 7).

Mutant Itgα6:

(SEQ ID NO: 1)

| | |
|---|---|
| probe: | TGGATCCCCCGGGCTGCA; |

(SEQ ID NO: 2)

| | |
|---|---|
| forward primer: | GAAACTGTAAAATTGACT AAATACCTTGCT; |

(SEQ ID NO: 3)

| | |
|---|---|
| reverse primer: | AGGGTTATTGAATATGATCGGAATTC. |
| Wildtype: | |

(SEQ ID NO: 4)

| | |
|---|---|
| Itgα6: probe: | CAAGGCTGAGATCCATACTCAGCCGTCTG; |

(SEQ ID NO: 5)

| | |
|---|---|
| forward primer: | AAAGATCATTACGATGCCACCTATC; |

(SEQ ID NO: 6)

| | |
|---|---|
| reverse primer: | GCTAGCGATGAAAAACATTGATCA. |
| Hoxa13: | |

(SEQ ID NO: 7)

| | |
|---|---|
| probe: | CAGCTGGTCCAGCACCCTCCCC; |

(SEQ ID NO: 8)

| | |
|---|---|
| forward primer: | TTACAGAAACAAAGGCTGTTTCCA; |

(SEQ ID NO: 9)

| | |
|---|---|
| reverse primer: | TGAGCAGGCACTTAACATGCA. |

PCR Detection of DNA Inversion in Photoreceptors
(FIG. 11)

(SEQ ID NO: 10)

| | |
|---|---|
| Primer A: | GTACGTCTTCCCGAGCGAAA. |

(SEQ ID NO: 11)

| | |
|---|---|
| Primer B: | CCCTGTGGCTGATCCTTG; |

(SEQ ID NO: 12)

| | |
|---|---|
| Primer C: | AGCTAGGGTGTACTGAGAATTTGG; |

(SEQ ID NO: 13)

| | |
|---|---|
| Primer D: | GTCATCGCTCTCCACACTCA. |

Real-Time PCR Quantification of DNA Inversion
(FIG. 11)

(SEQ ID NO: 14)

| | |
|---|---|
| Primer 1: | GCTACATCGACATGGTGTCCACTT; |

(SEQ ID NO: 15)

| | |
|---|---|
| Primer 2: | GTTGCTCCTACCTGGAAAGGATGA; |

(SEQ ID NO: 16)

| | |
|---|---|
| Primer 3: | TGTCCTTCTACCAGGGCTACACAA; |

(SEQ ID NO: 17)

| | |
|---|---|
| Primer 4: | TGTGCTGCAAGGCGATTAAGTTGG; |

(SEQ ID NO: 18)

| | |
|---|---|
| Primer 5: | CAGTACACCTGGCTGTTCCA; |

(SEQ ID NO: 19)

| | |
|---|---|
| Primer 6: | CAAACAAACAGTATGATCCCAGA. |

All published documents mentioned in the specification are herein incorporated by reference.

Various modifications and variations of the described invention will be apparent to those skilled in the art without departing from the scope and spirit of the invention. Although the invention has been described in connection with specific preferred embodiments, it should be understood that the invention as claimed should not be unduly limited to such specific embodiments. Various modifications of the above-described modes for carrying out the invention which are obvious to those skilled in the relevant fields of technology are intended to be within the scope of the following claims.

### REFERENCES

1. Branda, C. S. & Dymecki, S. M. (2004) Talking about a revolution: The impact of site-specific recombinases on genetic analyses in mice. Dev Cell 6:7-28;

2. Akeson, E. & Davisson M. (2000) Mitotic chromosome preparations from mouse cells for karyotyping. Current Protocols in Human Genetics 4.10.1-19;

3. Yu, Y. & Bradley, A. (2001) Engineering chromosomal rearrangements in mice. Nat Rev Genet 2:780-90;

4. Lewandoski, M. & Martin, G. R. (1997) Cre-mediated chromosome loss in mice. Nat Genet 17:223-5;

5. Matsumura, H., Tada, M., Otsuji, T., Yasuchika, K., Nakatsuji, N., Surani, A. & Tada, T. (2007) Targeted chromosome elimination from ES-somatic hybrid cells. Nat Methods 4:23-5;

6. Magnuson, T., Debrot, S., Dimpfl, J., Zweig, A., Zamora, T. & Epstein, C. J. (1985) The early lethality of autosomal monosomy in the mouse. J Exp Zool 236:353-60;

7. Kmita, M., Kondo, T. & Duboule, D. (2000) Targeted inversion of a polar silencer within the HoxD complex re-allocates domains of enhancer sharing. Nat Genet 26:451-4;

8. Logan, M., Martin, J. F., Nagy, A., Lobe, C., Olson, E. N. & Tabin, C. J. (2002) Expression of Cre Recombinase in the developing mouse limb bud driven by a Prxl enhancer. Genesis 33:77-80;

9. Novak, A., Guo, C., Yang, W., Nagy, A. & Lobe, C. G. (2000) Z/EG, a double reporter mouse line that expresses enhanced green fluorescent protein upon Cre-mediated excision. Genesis 28:147-55;,

10. Kmita, M., Fraudeau, N., Herault, Y. & Duboule, D. (2002) Serial deletions and duplications suggest a mechanism for the collinearity of Hoxd genes in limbs. Nature 420:145-50;

11. Danielian, P. S., Muccino, D., Rowitch, D. H., Michael, S. K. & McMahon, A. P. (1998) Modification of gene activity in mouse embryos in utero by a tamoxifen-inducible form of Cre recombinase. Curr Biol 8:1323-6;

12. Gimond, C., Baudoin, C., van der Neut, R., Kramer, D., Calafat, J. & Sonnenberg, A. (1998) Cre-loxP-mediated inactivation of the alpha6A integrin splice variant in vivo: evidence for a specific functional role of alpha6A in lymphocyte migration but not in heart development. J Cell Biol 143:253-66;

13. Le, Y. Z., Zheng, L., Zheng, W., Ash, J. D., Agbaga, M. P., Zhu, M. & Anderson, R. E. (2006) Mouse opsin promoter-directed Cre recombinase expression in transgenic mice. Mol Vis 12:389-98;

14. Bayani, J. & Squire J. (2004) Fluorescence in situ hybridization (FISH). Current Protocols in Cell Biology 22.4.1-52;

15. Young, R. W (1985) Cell differentiation in the retina of the mouse. Anat Rec. 212:199-205.

16. Gregoire, D., Kmita, M. (2008) Recombination between inverted loxP sites is cytotoxic for proliferating cells and provides a simple tool for conditional ablation. Proc Natl Acd Sci U.S.A. September 23; 105(38):14492-14496.

17. Mallet, V. O., Mitchell, C., Guidotti, J. E., Jaffray, P., Fabre, M., Spencer, D., Arnoult, D., Kahn, A., and Gilgenkrantz, H. (2002) Nat Biotechnol 20, 1234-9.

1. A method for causing cell death, the method comprising the step of manipulating chromosomal DNA of a cell to lose an autosome upon cell division, wherein manipulation of the chromosomal DNA involves a recombination event, and wherein loss of the autosome causes death of the cell.

2. The method of claim 1, wherein the autosome is chromosome 2.

3. The method of claim 1, wherein the autosome is engineered to carry a set of inverted recombinase recognition sequences and the cell carries a recombinase-encoding gene.

4. The method of claim 3, wherein the recombinase-encoding gene is under conditional expression.

5. The method of claim 3, wherein the conditional expression is achieved naturally.

6. The method of claim 3, wherein the conditional expression is achieved artificially.

7. The method of claim 3, wherein conditional expression of the recombinase is tissue-specific, development stage specific or cell type specific.

8. The method of claim 3, wherein the inverted recombinase recognition sequences are inverted loxP sites and the recombinase is Cre recombinase.

9. The method of claim 1, wherein the cell is a proliferating eukaryotic cell.

10. The method of claim 9, wherein the proliferating eukaryotic cell is a proliferating animal or plant cell.

11. A method for selective ablation of targeted cells within a population of cells, wherein the targeted cells comprises a set of inverted recombinase recognition sequences in an autosome and a recombinase-encoding gene, the method comprises expressing the recombinase in the targeted cells to lose the autosome during cell division resulting in ablation of the targeted cells.

12. The method of claim 11, wherein the autosome is chromosome 2.

13. The method of claim 11, wherein the recombinase is under conditional expression.

14. The method of claim 13, wherein the conditional expression is achieved naturally.

15. The method of claim 13, wherein the conditional expression is achieved artificially.

16. The method of claim 13, wherein conditional expression of the recombinase is tissue-specific, development stage specific or cell type specific.

17. The method of claim 11, wherein the inverted recombinase recognition sequences are inverted loxP sites and the recombinase is Cre recombinase.

18. The method of claim 11, wherein the targeted cells are proliferating eukaryotic cells.

19. The method of claim 18, wherein the proliferating eukaryotic cells are proliferating animal or plant cells.

20. A method of determining whether a selective ablation of targeted cells within an animal leads to a variation in the animal, the method comprising:
(a) obtaining a transgenic animal having targeted cells carrying a set of inverted recombinase recognition sequences in an autosome, and carrying a recombinase-encoding gene;
(b) expressing the recombinase within the targeted cells to lose the autosome during cell division, wherein loss of the autosome results in ablation of the targeted cells; and
(c) determining if there is a difference between the animal having a recombinase expressed in the targeted cells and a control animal not having the recombinase expressed in the targeted cells, wherein presence of a difference indicates that ablation of the targeted cells leads to a variation in the animal.

21. The method of claim 20, wherein the autosome is chromosome 2.

22. The method of claim 20, wherein the recombinase is under conditional expression.

23. The method of claim 22, wherein the conditional expression is achieved naturally.

24. The method of claim 22, wherein the conditional expression is achieved artificially.

25. The method of claim 22, wherein conditional expression of the recombinase is tissue-specific, development stage specific or cell type specific.

26 The method of claim 20, wherein the inverted recombinase recognition sequences are inverted loxP sites and the recombinase is Cre recombinase.

27. A method for producing a transgenic non-human organism having a targeted population of cells that have been ablated, the method comprising:
(a) producing an F1 generation by crossing a first and a second transgenic parent, the first transgenic parent carrying the set of inverted recombinase recognition sequences on an autosome, the second parent carrying a recombinase-encoding; and
(b) expressing the recombinase within targeted cells in an offspring of the F1 generation carrying the set of inverted recombinase recognition sequences and the recombinase-encoding gene;
wherein expression of the recombinase in the targeted cells results in loss of the autosome during cell division, causing ablation of the targeted population of cells within the offspring of the F1 generation defined in (b).

28. The method of claim 27, wherein the autosome is chromosome 2.

29. The method of claim 27, wherein the recombinase-encoding gene is under conditional expression.

30. The method of claim 27, further comprising the step of producing an F2 generation having a further targeted population of cells that have been ablated, comprising crossing the offspring of the F1 generation carrying a set of inverted recombinase recognition sequences and a first recombinase-encoding gene with a third transgenic parent carrying a second recombinase-encoding gene, whereas the first and the second recombinase-encoding genes are expressed under different conditional expressions; expressing said first and second recombinases within their respective targeted cells in an offspring of the F2 generation carrying the set of inverted recombinase recognition sequences and said first and second recombinase-encoding genes; wherein expression of said recombinases in the targeted cells results in loss of the autosome during cell division, causing ablation of targeted cells within the offspring of the F2 generation.

31. The method of claim 27, wherein the inverted recombinase recognition sequences are inverted loxP sites and the recombinase is Cre recombinase.

32. The method of claim 27, wherein the targeted cells are proliferating cells.

33. The method of claim 27, wherein the non-human organism is a eukaryotic organism.

34. The method of claim 33, wherein the eukaryotic organism is a non-human animal or a plant.

35. The method of claim 34, wherein the non-human animal is a mouse or a rat.
